(19) United States
(12) Patent Application Publication
(10) Pub. No.: US 2022/0323016 A1
(43) Pub. Date: Oct. 13, 2022

(54) BIOSIGNAL MEASURING APPARATUS, BIOSIGNAL PROCESSING APPARATUS AND METHOD OF OPERATING BIOSIGNAL PROCESSING APPARATUS

(71) Applicant: ATSENS CO., LTD., Gyeonggi-do (KR)

(72) Inventors: Kab Mun CHA, Seoul (KR); Jong Sung KIM, Seoul (KR); Jong Ook JEONG, Gyeonggi-do (KR)

(73) Assignee: ATSENS CO., LTD., Gyeonggi-do (KR)

(21) Appl. No.: 17/706,059

(22) Filed: Mar. 28, 2022

(30) Foreign Application Priority Data
Apr. 13, 2021 (KR) 10-2021-0048020

(51) Int. Cl. A61B 5/00; A61B 5/282; A61B 5/318; G01R 27/02; A61B 5/332

(52) U.S. Cl. CPC A61B 5/7203; A61B 5/282; A61B 5/318; G01R 27/02; A61B 5/332

(57) ABSTRACT

A biosignal measuring apparatus, a biosignal processing apparatus, and an operating method of the biosignal processing apparatus are provided. The biosignal measuring apparatus may include a plurality of electrodes configured to be in contact with a skin of an object and receive electrical signals generated from the object, a biosignal sensing circuit electrically connected to the plurality of electrodes and configured to generate a biosignal based on the electrical signals received through the plurality of electrodes, an impedance measuring circuit electrically connected to the plurality of electrodes and configured to measure an impedance between the plurality of electrodes which is to be used for correcting a magnitude of the biosignal varying with time, and a signal processing unit configured to receive the biosignal from the biosignal sensing circuit and an impedance value from the impedance measuring circuit, and correct the magnitude of the biosignal based on the impedance value.

### CROSS-REFERENCE TO RELATED APPLICATION

This application is based on and claims priority under 35 U.S.C. § 119 to Korean Patent Application No. 10-2021-0048020, filed on Apr. 13, 2021, in the Korean Intellectual Property Office, the disclosure of which is incorporated by reference herein in its entirety.

### BACKGROUND

### 1. Field

One or more embodiments relates to a biosignal measuring apparatus capable of measuring the impedance between a plurality of electrodes used to receive electrical signals from an object, a biosignal processing apparatus configured to process a biosignal and an impedance value received from the biosignal measuring apparatus, and a method of operating the biosignal processing apparatus.

### 2. Description of the Related Art

Imaging tests are used in addition to clinical examinations to examine the presence of abnormalities in the heart. As an early diagnosis method, a method of measuring an electrocardiogram and determining the presence of abnormalities in the heart of a patient based on the measured electrocardiogram is also widely used. An electrocardiogram refers to a graph in which potential variations on the body surface are recorded according to the mechanical activity of heartbeat such as contraction or expansion of the heart muscle. Electrocardiography is a noninvasive test that is simple in measurement, reproducible, easily repeatable, and inexpensive and is widely used for diagnosing arrhythmia and coronary artery disease (cardiovascular disease) and monitoring cardiac patients.

An electrocardiogram is measured by attaching electrocardiogram-measuring electrodes to an object, for example, the upper left and right sides and the lower left and right sides of the chest of a human, and measuring a potential difference between the positions of the electrocardiogram-measuring electrodes. Long-term electrocardiogram monitoring is required for accurate diagnosis of cardiac abnormalities. During daily activities of a user in a state in which electrodes are attached to a user, sweat or external moisture may permeate between the body and the electrodes. In this case, the impedance between the electrodes may vary, and thus the electrocardiogram of the user may vary with time.

### SUMMARY

One or more embodiments include a biosignal measuring apparatus configured to measure the impedance between electrodes for measuring an electrocardiogram signal, a biosignal processing apparatus configured to correct the magnitude of the electrocardiogram signal based on a measured impedance value, and a method of operating the biosignal processing apparatus.

One or more embodiments include a biosignal processing apparatus capable of early determining a health state based on the magnitude of an electrocardiogram signal, and a method of operating the biosignal processing apparatus.

Additional aspects will be set forth in part in the description which follows and, in part, will be apparent from the description, or may be learned by practice of the presented embodiments of the disclosure.

According to one or more embodiments, a biosignal measuring apparatus includes: a plurality of electrodes configured to be in contact with a skin of an object and receive electrical signals generated from the object; a biosignal sensing circuit electrically connected to the plurality of electrodes and configured to generate a biosignal based on the electrical signals received through the plurality of electrodes; an impedance measuring circuit electrically connected to the plurality of electrodes and configured to measure an impedance between the plurality of electrodes which is to be used for correcting a magnitude of the biosignal varying with time; and a signal processing unit configured to receive the biosignal from the biosignal sensing circuit and an impedance value from the impedance measuring circuit, and correct the magnitude of the biosignal based on the impedance value.

According to one or more embodiments, a biosignal processing apparatus includes: a receiving unit configured to receive, from a biosignal measuring apparatus, a biosignal generated based on electrical signals received through a plurality of electrodes attached to an object and an impedance value between the plurality of electrodes; and a biosignal correcting unit configured to generate a corrected biosignal by correcting a magnitude of the biosignal based on the impedance value.

According to one or more embodiments, a method of operating a biosignal processing apparatus includes: receiving, from a biosignal measuring apparatus, a biosignal generated based on electrical signals received through a plurality of electrodes attached to a skin of an object and an impedance value between the plurality of electrodes; and generating a corrected biosignal by correcting a magnitude of the biosignal based on the impedance value.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a view illustrating a biosignal measuring apparatus attached to an object according to an example embodiment;

FIG. 2 is a block diagram schematically illustrating a biosignal measuring apparatus according to an example embodiment;

FIG. 3 is a view illustrating a sensor unit and a signal processing unit of a biosignal measuring apparatus according to an example embodiment;

FIG. 4A is a view schematically illustrating an example of a biosignal sensing circuit according to an example embodiment;

FIG. 4B is a view schematically illustrating an example of an impedance measuring circuit according to an example embodiment;

FIG. 5A is a view illustrating a state in which a plurality of electrodes are attached to the skin surface of an object in order to sense an electrocardiogram of the object;

FIG. 5B is a view illustrating an equivalent model of impedances shown in FIG. 5A;

FIG. 6 is a graph illustrating variations in an electrocardiogram signal with respect to time;

FIG. 7A is a graph illustrating the magnitude of an electrocardiogram signal and the magnitude of a corrected electrocardiogram signal according to an example embodiment;

FIG. 7B is a graph illustrating the magnitude of an electrocardiogram signal, the magnitude of a corrected electrocardiogram signal, and an impedance value according to an example embodiment;

FIG. 8 is a block diagram illustrating a biosignal monitoring system according to an example embodiment;

FIG. 9 is a flowchart illustrating a method of operating a biosignal processing apparatus according to an example embodiment;

FIG. 10 is a flowchart illustrating a method of determining a health state according to an example embodiment;

FIG. 11 is a flowchart illustrating a method of determining a health state according to an example embodiment;

FIG. 12A illustrates a biosignal monitoring system according to one or more embodiments; and

FIG. 12B illustrates a biosignal monitoring systems according to another embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Therefore, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Expressions such as “includes” or “may include” used in various embodiments of the present disclosure specify the presence of stated functions, operations, or elements, but do not preclude the presence or addition of one or more other functions, operations, or elements. In addition, the meaning of “include” or “comprise” specifies a property, a fixed number, a step, a process, an element, a component, and a combination thereof but does not exclude one or more other properties, fixed numbers, steps, processes, elements, components, and combinations thereof.

In various embodiments, expressions such as “or” include any and all combinations of words listed together. For example, “A or B” may refer to A, or may refer to B, or may refer to both A and B.

Expressions such as “first” and “second” may be used in various embodiments to describe various elements, but do not limit the elements. For example, the expressions do not specify the order and/or importance of elements. The expressions may be used to distinguish one element from another. For example, a first user device and a second user device are user devices different from each other. For example, without departing from the scope of various embodiments, a first element may be referred to as a second element, and similarly, a second element may also be referred to as a first element.

It should be understood that when an element is referred to as being “coupled,” or “connected,” to another element, the element may be coupled or connected directly to the other element or any other element may be interposed between the two elements. In contrast, it may be understood that when an element is referred to as being “directly coupled,” or “directly connected” to another element, there is no element interposed between the two elements.

In embodiments, terms such as “module,” “unit,” or “part” may be used to denote an element that has at least one function or operation and is implemented with hardware, software, or a combination of hardware and software. In addition, a plurality of “modules,” “units,” or “parts” may be integrated into at least one module or chip as at least one processor, except when each needs to be implemented as an individual specific hardware element.

Terms used herein are merely for the purpose of describing particular embodiments and are not intended to limit the scope of other embodiments. As used herein, singular forms may include plural forms as well unless the context clearly indicates otherwise.

Unless defined otherwise, all terms used herein, including technical and scientific terms, have the same meaning as those commonly understood by a person skilled in the art to which the present disclosure pertains.

Terms such as those defined in a generally used dictionary may be interpreted to have the same meanings as the contextual meanings in the relevant field of art, and are not to be interpreted to have ideal or excessively formal meanings unless clearly defined herein.

Hereinafter, various embodiments will be described in detail with reference to the accompanying drawings.

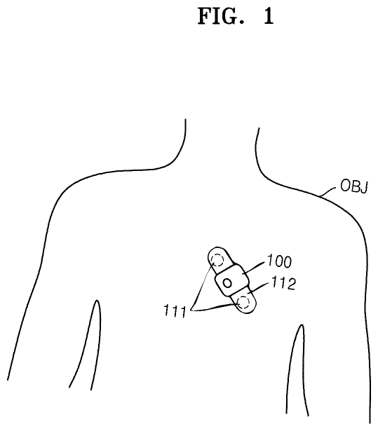

FIG. 1 illustrates a biosignal measuring apparatus 100 attached to an object according to an example embodiment.

The biosignal measuring apparatus 100 is attached to the object OBJ in a non-invasive or invasive manner to sense a biosignal of the object. Referring to FIG. 1, the biosignal measuring apparatus 100 may be an electrocardiogram signal measuring apparatus attached to the chest of the object OBJ to detect or measure an electrocardiogram according to the heartbeat of the object OBJ. Here, the object OBJ may be a part of a human or animal body, such as the chest of the human or animal, but is not limited thereto. The object OBJ may be any object from which an electrocardiogram may be sensed or measured. In addition, the term “electrocardiogram” refers to a graph that records potential variations appearing on the body surface according to mechanical heartbeat activities such as the contraction/expansion of the myocardium. The expression “sensing an electrocardiogram” has the same meaning as the expression “sensing an electrical potential” generated on the body surface according to the heartbeat of the object OBJ.

The biosignal measuring apparatus 100 may transmit/receive data to/from a user terminal through a communication module. The communication module may include various communication modules such as a wireless Internet module, a short-range communication module, or a mobile communication module.

The biosignal measuring apparatus 100 may include a plurality of electrodes 111, and the plurality of electrodes 111 may receive electrical signals generated on the object OBJ. The biosignal measuring apparatus 100 may generate a biosignal and an impedance value based on the electrical signals received through the plurality of electrodes 111.

The biosignal measuring apparatus 100 may further include band-type mounting portions 112, and the mounting portions 112 may include a flexible material such as an elastic or stretchable fabric which is deformable according to the curvature of the body surface. The mounting portions 112 may be provided as a patch type or a wear type. Owing to the mounting portions 112, the plurality of electrodes 111 may be brought into contact with the body surface of the object OBJ to sense a potential generated on the body surface of the object OBJ.

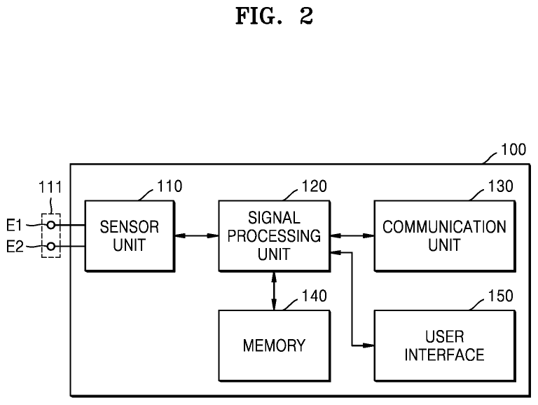

FIG. 2 is a block diagram schematically illustrating a biosignal measuring apparatus 100 according to an example embodiment.

Referring to FIG. 2, the biosignal measuring apparatus 100 may include: a plurality of electrodes 111, a sensor unit 110, a signal processing unit 120, a communication unit 130, and a memory 140. The biosignal measuring apparatus 100 may further include a user interface 150. In addition, the biosignal measuring apparatus 100 may further include general-purpose components for an electronic apparatus, such as a power supply unit or a clock signal generator.

The biosignal measuring apparatus 100 may be an apparatus for measuring a biosignal of a person or an animal. For example, the biosignal may be one of signals indicating a body temperature, a pulse rate, an electrocardiogram, a brain wave, an electromyogram, a respiration rate, a step count, stress, hormone, an exercise amount, calories burned, body fat, a body water content, a blood sugar value, a blood pressure, etc. Hereinafter, an example of the present disclosure, in which a biosignal refers to an electrocardiogram signal, and the biosignal measuring apparatus 100 is an electrocardiogram measuring apparatus, will be described.

As described with reference to FIG. 1, the biosignal measuring apparatus 100 may be mounted on an object (refer to the object OBJ in FIG. 1) in a non-invasive or invasive manner to measure an electrocardiogram according to the heartbeat of the object.

The plurality of electrodes 111 may be attached to the skin surface of the object to receive electrical signals of two or more channels generated from the object OBJ. In the current embodiment, the plurality of electrodes 111 may include a first electrode E1 and a second electrode E2. However, embodiments are not limited thereto, and the plurality of electrodes 111 may include three or more electrodes.

The plurality of electrodes 111 are electrocardiogram electrodes for measuring an electrocardiogram. The plurality of electrodes 111 may be receive electrocardiographic electrical signals by inducing an action current on the body surface, which is generated in the myocardium according to the heartbeat of the object.

The sensor unit 110 may sense electrical signals of two or more channels received through the plurality of electrodes 111 electrically connected to the sensor unit 110. The sensor unit 110 may generate a biosignal, that is, an electrocardiogram signal, based on the electrical signals.

In an embodiment, the sensor unit 110 may generate an impedance value by measuring the impedance between the plurality of electrodes 111, for example, between the first electrode E1 and the second electrode E2 as well as the sensor unit 110 generating a biosignal.

Long-term electrocardiogram monitoring is required for accurate diagnosis of cardiac abnormalities. The sensor unit 110 may generate a biosignal and an impedance value during a monitoring period, that is, during the entire period of sensing. In this case, external moisture, or sweat or oil generated from the object while the plurality of electrodes 111 are on the object may be introduced between the body and the plurality of electrodes 111. As a result, the impedance between the plurality of electrodes 111 may be changed, and the magnitude of the biosignal may be changed with time. To correct the magnitude of the biosignal that changes with time, the sensor unit 110 may measure the impedance between the plurality of electrodes 111, and an impedance value obtained by the impedance measurement may be used to correct the magnitude of the biosignal.

The signal processing unit 120 may be electrically connected to the sensor unit 110, the communication unit 130, and the memory 140 to process a biosignal such as an electrocardiogram signal of the object and store data obtained by processing the biosignal, that is, biometric data, in the memory 140 or transmit the biometric data to an external receiving device through the communication unit 130.

For example, the signal processing unit 120 may convert the electrocardiogram signal to reduce power consumption by considering the power capacity of the biosignal measuring apparatus 100, or may convert the electrocardiogram signal to adjust the amount of transmission data by considering transmission capability. The signal processing unit 120 may generate information indicating the heart state of the object based on the biometric data.

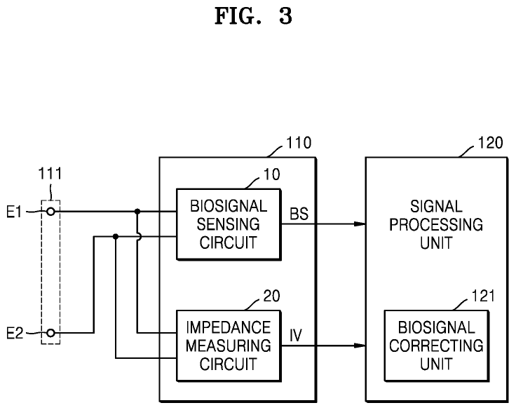

In an embodiment, the signal processing unit 120 may receive a biosignal and an impedance value from the sensor unit 110 and correct the biosignal based on the impedance value. The signal processing unit 120 may correct the magnitude of the biosignal, which changes with time, based on the impedance value measured in the same period as the period in which the biosignal is measured. This will be described later with reference to FIGS. 3 to 7.

In an embodiment, the signal processing unit 120 may control the memory 140 and the communication unit 130 such that the biosignal and the impedance value may be transmitted to an external device, for example, a biosignal processing apparatus, and the biosignal processing apparatus may correct the magnitude of the biosignal based on the impedance value.

The signal processing unit 120 may be implemented as at least one processor, and for example, the processor may execute various functions and programs stored in the memory 140 of the biosignal measuring apparatus 100 to control the overall operation of the biosignal measuring apparatus 100. The processor may include at least one selected from the group consisting of a digital signal processing unit (DSP), a microprocessor, a central processing unit (CPU), a micro controller unit (MCU), a micro processing unit (MPU), a controller, an application processor (AP), a communication processor (CP), and an ARM processor, or may be defined by a corresponding term. In addition, the processor may be implemented as a system on chip (SoC), a large scale integration (LSI) processor, or a field programmable gate array (FPGA), which has a processing algorithm therein.

The communication unit 130 may transmit/receive data to/from devices such as a server and other electronic devices through a communication network. The communication unit 130 may transmit/receive data through a wireless network or a wired network. The communication unit 130 may process and transmit data under the control of the signal processing unit 120. In an embodiment, the communication module 130 may include various communication modules such as a wireless Internet module, a short-range communication module, or a mobile communication module.

The wireless Internet module refers to a communication module connected to an external network according to a communication protocol such as wireless LAN (WLAN), Wi-Fi, wireless broadband (Wibro), world interoperability for microwave access (Wimax), or high speed downlink packet access (HSDPA).

The short-range communication module refers to a module for short-range communication with an external device according to a short-range communication protocol such as Bluetooth, radio frequency identification (RFID), infrared communication (IrDA), ultra wideband (UWB), or ZigBee.

The mobile communication module refers to a communication module connected to a mobile communication network according to various mobile communication protocols such as 3rd generation (3G), 3rd generation partnership project (3GPP), or long term evolution (LTE).

However, embodiments are not limited thereto, and the communication unit 130 may include a communication module other than the communication modules described above as long as the biosignal measuring apparatus 100 may transmit/receive various signals and data to/from external devices through the communication unit 130.

The memory 140 may store biometric data including electrocardiogram signals and impedance values which are sensed or measured by the sensor unit 110. The memory 140 may store programs such that the signal processing unit 120 may perform processing operations and control operations with the programs. The memory 140 may store data to be transmitted through the communication unit 130 and data received through the communication unit 130.

The user interface 150 may include a manipulation unit that receives a user's command and a display unit that displays information related to an operating state of the biosignal measuring apparatus 100. The user interface 150 may generate a command corresponding to a user's manipulation, for example, a power-on or power-off command, and may output the generated command to the signal processing unit 120. The manipulation unit may include at least one of various input devices such as a physical button, an optical key, a keypad, or a voice input device. The display unit may display the operating state of the biosignal measuring apparatus 100 under the control of the signal processing unit 120. The display unit may provide various information regarding the operation of the biosignal measuring apparatus 100 by a visual method, an auditory method, or a method using other senses. To this end, the display unit may include a display unit and/or a speaker. For example, when the electrodes 111 are separated from the skin of the object, the display unit may output a corresponding warning signal under the control of the signal processing unit 120. In an embodiment, the manipulation unit and the display unit of the user interface 150 may be implemented as one device. For example, the user interface 150 may be implemented as a touch display in which a manipulation unit and a display unit are combined with each other.

The biosignal measuring apparatus 100 may further include various sensors that collect biometric information different from biometric information collected by the sensor unit 110. The biosignal measuring apparatus 100 may further include a motion sensor, a blood pressure sensor, a heart rate sensor, or the like.

In addition, although the sensor unit 110 and the signal processing unit 120 are provided in one device in the embodiment shown in FIG. 1, the sensor unit 110 and the signal processing unit 120 may be provided and implemented in separate devices. In this case, the sensor unit 110 and the signal processing unit 120 may be electrically connected to each other or may connected to each other through a communication network.

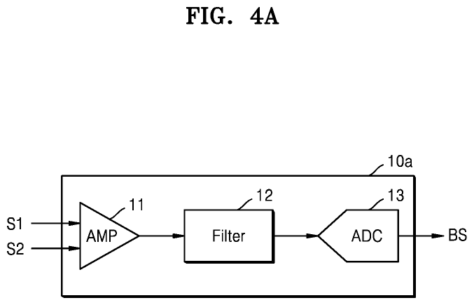

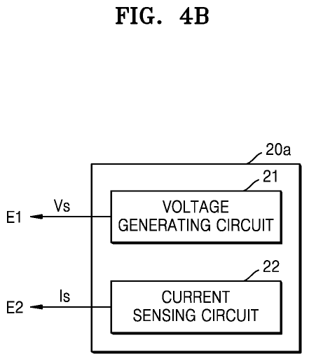

FIG. 3 illustrates a sensor unit 110 and a signal processing unit 120 of a biosignal measuring apparatus according to an example embodiment. FIG. 4A schematically illustrates by example a biosignal sensing circuit 10a according to an example embodiment. FIG. 4B schematically illustrates by example an impedance measuring circuit 20a according to an example embodiment.

Referring to FIG. 3, the sensor unit 110 may include a biosignal sensing circuit 10 and an impedance measuring circuit 20.

The biosignal sensing circuit 10 may be electrically connected to a plurality of electrodes 111 and may generate a biosignal BS such as an electrocardiogram signal based on electrical signals received through the plurality of electrodes 111.

Referring to FIG. 4A, the biosignal sensing circuit 10a according to an embodiment may include an amplifier 11, a filter 12, and an analog-to-digital converter (ADC) 13. In an embodiment, the biosignal sensing circuit 10a may further include a programmable gain amplifier.

The amplifier 11 may amplify and output the difference between received signals such as a first signal S1 received through a first electrode E1 and a second signal E2 received through a second electrode E2. For example, the first signal S1 and the second signal S2 may be voltage signals, and the amplifier 11 may output an amplified voltage signal. The amplifier 11 may be implemented as a differential amplifier.

The filter 12 may remove low-frequency or high-frequency noise from the amplified voltage signal. The analog-to-digital converter 13 may convert the voltage signal into digital values and may output the digital values as a biosignal BS.

Referring to FIG. 3, the impedance measuring circuit 20 may be electrically connected to the plurality of electrodes 111, and may measure impedance between the plurality of electrodes 111, for example, between the first electrode E1 and the second electrode E2. The impedance measuring circuit 20 may periodically or aperiodically measure the impedance and generate an impedance value according to the measured impedance. The impedance measuring circuit 20 may measure impedance corresponding to a frequency band of the biosignal BS.

Referring to FIG. 4B, the impedance measuring circuit 20a according to an example embodiment may include a voltage generating circuit 21 and a current sensing circuit 22.

The voltage generating circuit 21 may generate a sensing voltage Vs and may apply the sensing voltage Vs to one of the plurality of electrodes 111, for example, the first electrode E1. In an embodiment, the sensing voltage Vs may be a pulse voltage having a given frequency and magnitude. The frequency of the sensing voltage Vs may be included in the frequency band of the biosignal BS.

The current sensing circuit 22 may receive a sensing current Is, which is generated as the sensing voltage Vs is applied through another of the plurality of electrodes 111, for example, through the second electrode E2.

The impedance measuring circuit 20a may measure impedance by calculating the impedance between the first electrode E1 and the second electrode E2 based on the sensing voltage Vs and the sensing current Is. The impedance measuring circuit 20a may output an impedance value corresponding to the measured impedance.

Although the biosignal sensing circuit 10a and the impedance measuring circuit 20a have been described as examples with reference to FIGS. 4A and 4B, embodiments are not limited thereto. For example, the biosignal sensing circuit 10a and the impedance measuring circuit 20a may be implemented with other components or circuits.

Referring to FIG. 3, the signal processing unit 120 may include a biosignal correcting unit 121. The biosignal correcting unit 121 may receive a biosignal BS and an impedance value IV from the sensor unit 110 and may correct the biosignal BS based on the impedance value IV.

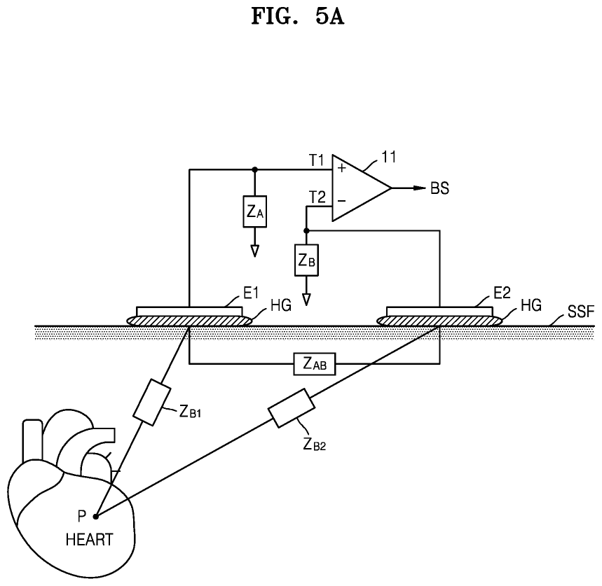

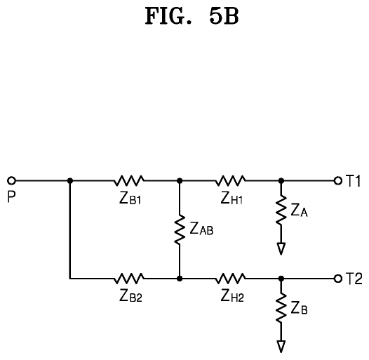

FIG. 5A illustrates a state in which a plurality of electrodes are attached to the skin surface of an object to sense an electrocardiogram of the object. FIG. 5B illustrates an equivalent model of impedances shown in FIG. 5A.

It is assumed that an amplifier 11 is ideal, and an input impedance of the amplifier 11 is infinite.

Referring to FIG. 5A, the surfaces of the plurality of electrodes, for example, a first electrode E1 and a second electrode E2, may be coated with hydrogel HG, and the first electrode E1 and the second electrode E2 may be attached to a skin surface SSF of the object through the hydrogel HG. The first electrode E1 and the second electrode E2 may be connected to the amplifier 11 provided in a biosignal sensing circuit (refer to the biosignal sensing circuit 10 shown in FIG. 3), and electrical signals received through the first electrode E1 and the second electrode E2 may be provided to the amplifier 11. The amplifier 11 may amplify, as an amplified signal, the difference between the electrical signals received through the first electrode E1 and the second electrode E2, and the amplified signal may be converted into a digital signal as a biosignal BS.

Impedances Z_{B1} and Z_{B2} are impedances between a point P of the heart, for example, a point at which an electrocardiogram signal is fired, and a skin surface SSF to which the first electrode E1 and the second electrode E2 are attached. The impedances Z_{B1} and Z_{B2} may vary depending on the distance between the point P and the points to which the first electrode E1 and the second electrode E2 are attached, anatomical factors (for example, bones, blood vessels, etc.), fat, water, etc.

Impedances Z_{H1} and Z_{H2} refer to the impedance of the hydrogel HG. The impedances Z_{H1} and Z_{H2} may be determined by the thickness of the hydrogel HG and may vary depending on electrical components of the hydrogel HG which may vary due to skin secretions (for example, sweat, fat, etc.).

Impedance Z_{AB} refers to the impedance between the first electrode E1 and the second electrode E2 and may include the impedance of the skin surface SSF between the first electrode E1 and the second electrode E2 and the internal impedance of the body between the first electrode E1 and the second electrode E2. The impedance Z_{AB} may vary depending on skin secretions (for example, sweat, fat, etc.), and the variation of the impedance Z_{AB} may be greater than the variations of the impedances Z_{H1} and Z_{H2}.

Impedance Z_{A} may refer to a shunt impedance of the first electrode E1 and a first input terminal T1 of the amplifier 11, and impedance ZB may refer to a shunt impedance of the second electrode E2 and a second input terminal T2 of the amplifier 11.

After the first electrode E1 and the second electrode E2 are attached to the skin surface SSF of the object, skin secretions (for example, sweat, fat, etc.) may be generated on the skin surface SSF over time, and external moisture may also permeate between the skin surface SSF and the first and second electrodes E1 and E2. Therefore, the impedances Z_{H1} and Z_{H2} and the impedance Z_{AB} may vary over time.

Variations in the impedances Z_{H1} and Z_{H2} and the impedance Z_{AB} may cause variations in the amplitude of an amplified voltage signal output from the amplifier 11. In other words, the amplitude of a biosignal BS may vary. The impedance Z_{AB} may be greater than the impedances Z_{H1} and Z_{H2}, and as time passes, variations in the amplitude of the impedance Z_{AB} may have a dominant effect on variations in the amplitude of the biosignal BS.

Therefore, to compensate for variations in the amplitude of the biosignal BS over time, the impedance measuring circuit 20 may measure the impedance Z_{AB}, and the biosignal BS may be corrected based on a measured impedance value.

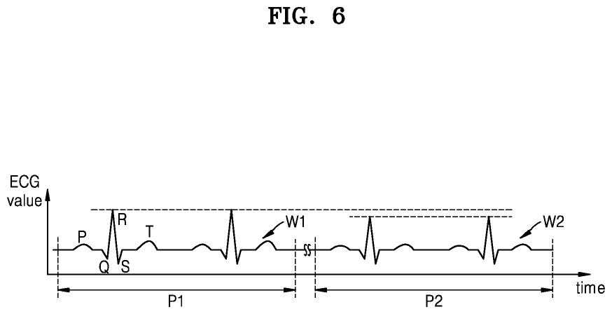

FIG. 6 illustrates an electrocardiogram signal with respect to time.

The horizontal axis refers to time, and the vertical axis refers to an electrocardiogram (ECG) signal value.

Referring to FIG. 6, the electrocardiogram signal may include P, Q, R, S, and T waves which occur repetitively.

A biosignal measuring apparatus (for example, the biosignal measuring apparatus 100 shown in FIG. 1) such as an electrocardiogram signal measuring apparatus may be attached to an object and maintained on the object for a long period of time (for example, 14 days) to sense an electrocardiogram signal. During the sensing period, an electrocardiogram waveform in a first period P1, for example, a first waveform W1, may be different from an electrocardiogram waveform in a second period P2, for example, a second waveform W2. For example, the peak value of the R wave of the first waveform W1 may be greater than the peak value of the R wave of the second waveform W2. In another example, the value of the first waveform W1 may be greater than the value of the second waveform W2 as a whole. The magnitude of an electrocardiogram signal may be calculated for each period. In a non-limiting example, the average of the absolute values of the peak values of the R waves in respective periods, or the average of the absolute values of P waves, Q waves, R waves, or S waves in the periods may be calculated as the magnitude of the electrocardiogram signal for the periods.

Therefore, the magnitude of an electrocardiogram signal may vary with time. For example, the magnitude of the electrocardiogram signal in the first period P1, that is, the magnitude of the first waveform W1, may be different from the magnitude of the electrocardiogram signal in the second period P2, that is, the magnitude of the second waveform W2.

In the embodiment shown in FIG. 6, each of the first period P1 and the second period P2 includes two P waves, two Q waves, two R waves, two S waves, and two T waves. However, embodiments are not limited thereto, and periods may be set in various ways. For example, a period between the peak values of R waves, that is, an R-R interval, may be set as a period, and in this case, the lengths of periods may be different from each other.

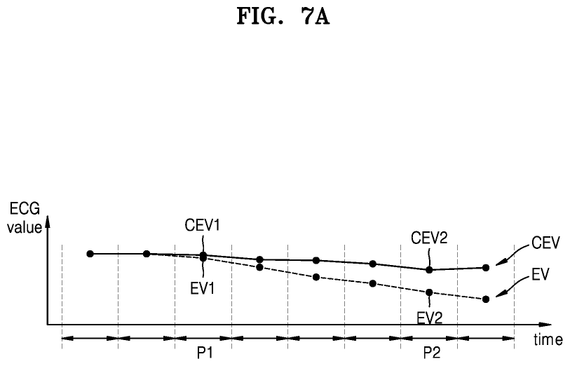

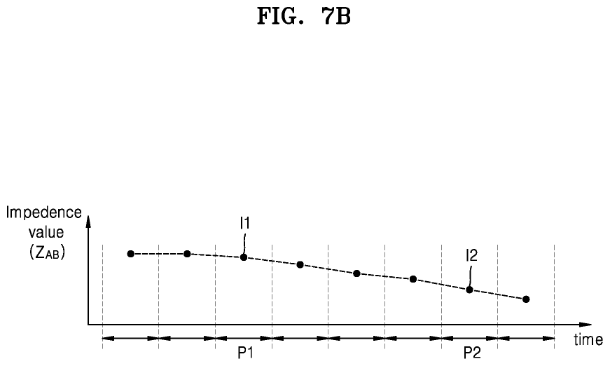

FIGS. 7A and 7B illustrate an electrocardiogram signal magnitude, a corrected electrocardiogram signal magnitude, and an impedance value according to an example embodiment.

In FIG. 7A, the horizontal axis refers to time, and the vertical axis refers to an electrocardiogram signal value. EV refers to an electrocardiogram signal magnitude value with time, and CEV refers to a corrected electrocardiogram signal magnitude value with time. In FIG. 7B, the horizontal axis refers to time, and the vertical axis refers to an impedance value. The impedance value refers to impedance Z_{AB} between a plurality of electrodes, for example, a first electrode (see the first electrode E1 in FIG. 2) and a second electrode (see the second electrode E2 in FIG. 2) which are attached to the skin surface of an object for sensing an electrocardiogram signal.

Referring to FIG. 7A, the electrocardiogram signal magnitude value EV may decrease with time, and this decrease is because of variations in the impedance Z_{AB} between the plurality of electrodes as shown in FIG. 7B.

A biosignal correcting unit (such as the biosignal correcting unit 121 in FIG. 3) may correct the electrocardiogram signal magnitude value EV based on the value of the impedance Z_{AB} between the plurality of electrodes for each period, and thus the corrected electrocardiogram signal magnitude value CEV may be calculated.

For example, a first magnitude value EV1 of an electrocardiogram signal may be corrected based on a first impedance value I1 in a first period P1 to calculate a first corrected magnitude value CEV1 of the electrocardiogram signal. In addition, a second magnitude value EV2 of the electrocardiogram signal may be corrected based on a second impedance value I2 in a second period P2 to calculate a second corrected magnitude value CEV2 of the electrocardiogram signal. Similarly, the corrected electrocardiogram signal magnitude value CEV may be calculated for each period.

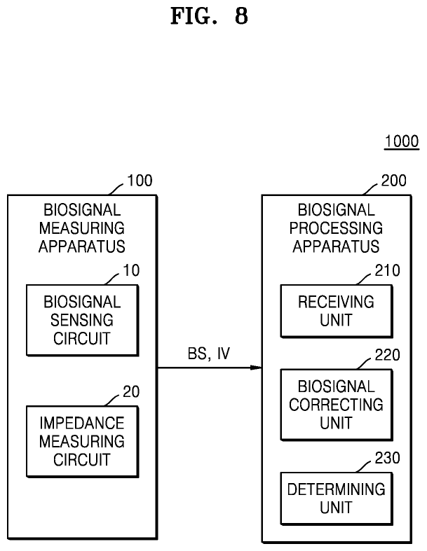

FIG. 8 is a block diagram illustrating a biosignal monitoring system 1000 according to an example embodiment.

Referring to FIG. 8, the biosignal monitoring system 1000 may include a biosignal measuring apparatus 100 and a biosignal processing apparatus 200. The biosignal measuring apparatus 100 and the biosignal processing apparatus 200 may transmit and receive data by a wired or wireless communication method.

The biosignal measuring apparatus 100 shown in FIG. 2 may be used as the biosignal measuring apparatus 100 shown in FIG. 8, and therefore, the description of the biosignal measuring apparatus 100 shown in FIG. 2 may be applied to the current embodiment.

The biosignal measuring apparatus 100 may include: a biosignal sensing circuit 10 configured to generate a biosignal BS; and an impedance measuring circuit 20 configured to generate an impedance value IV by measuring the impedance between a plurality of electrodes which are used to sense the biosignal BS. The biosignal measuring apparatus 100 may transmit the biosignal BS and the impedance value IV to the biosignal processing apparatus 200.

The biosignal processing apparatus 200 may include a receiving unit 210, a biosignal correcting unit 220, and a determining unit 230.

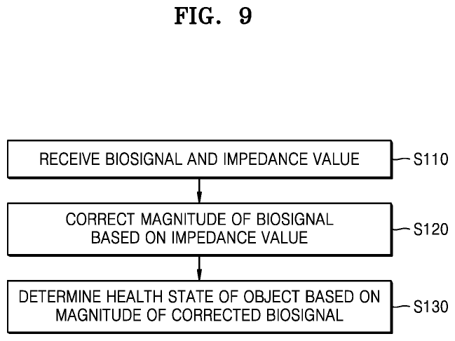

FIG. 9 is a flowchart illustrating a method of operating the biosignal processing apparatus 200 according to an example embodiment. The method of operating the biosignal processing apparatus 200 will now be described with reference to FIGS. 8 and 9 together.

The receiving unit 210 may receive a biosignal BS and an impedance value IV from the biosignal measuring apparatus 100 (S110). The receiving unit 210 may be implemented as a wired or wireless communication module. The receiving unit 210 may receive a biosignal BS and an impedance value IV from the biosignal measuring apparatus 100 in real time (or with a delay) by a wired or wireless communication method, or may receive a biosignal BS and an impedance value IV, which correspond to the entire sensing period, from the biosignal measuring apparatus 100. For example, the biosignal measuring apparatus 100 may store, in an internal device (for example, a memory), a biosignal BS and an impedance value IV generated in real time during a sensing period, and after the sensing period, the biosignal measuring apparatus 100 may transmit, to the interconnector 200, the biosignal BS and the impedance value IV corresponding to the entire sensing period.

The biosignal correcting unit 220 may correct the magnitude of the biosignal BS based on the impedance value IV (S120). The method of correcting the magnitude of the biosignal BS based on the impedance value IV has been described with reference to FIG. 7, and thus a description thereof will not be repeated here.

The determining unit 230 may determine the health state of an object based on the magnitude of the corrected biosignal (S130). The determining unit 230 may determine the health state of the object by correlating the magnitude of the corrected biosignal BS with other biological information on the object.

For example, the biological information on the object may be fixed biological information such as the height, weight, body fat, age, or gender of the object, which does not vary during the sensing period of the biosignal BS. The biosignal processing apparatus 200 may receive the fixed biological information through the receiving unit 210 or a separate input unit.

In another example, the biological information may be variable biological information such as the heart rate, breathing rate, water content, blood pressure, or activity state of the object, which varies during the sensing period of the biosignal BS. The biosignal processing apparatus 200 may receive fixed biological information through the receiving unit 210 or a separate input unit, and may obtain variable biological information from the biosignal measuring apparatus 100 or another measuring apparatus. For example, the biosignal measuring apparatus 100 may further include a motion sensor. The biosignal measuring apparatus 100 may generate activity state information based on motion information sensed using the motion sensor, and may provide the activity state information to the biosignal processing apparatus 200.

Based on the magnitude of the corrected biosignal BS and other biological information, the determining unit 230 may determine that the object probably has a health problem or may determine various health states of the object. For example, the determining unit 230 may determine whether it is necessary for the object to have a medical checkup or regular exercise for health improvements.

The determining unit 230 may be implemented as a machine learning device, an offline server, or the like, and may statistically determine the health state of the object based on the magnitude of the corrected biosignal BS and other biological information. The determining unit 230 may model a health state determination algorithm through training with training data such as various pieces of fixed biological information and variable biological information on other objects. The determining unit 230 may generate a model through training with the training data, and the health state determination algorithm may be trained by a method such as a supervised learning, unsupervised learning, or reinforcement learning method. The health state determination algorithm may be generated by an algorithm such as a decision tree, a Bayesian network, a support vector machine, or an artificial neural network (ANN).

In the current embodiment, the biosignal processing apparatus 200 includes the biosignal correcting unit 220. However, embodiments are not limited thereto. In other embodiments, the biosignal correcting unit 220 may be provided in the biosignal measuring apparatus 100, and the biosignal processing apparatus 200 may receive a biosignal BS corrected based on an impedance value IV and may determine the health state of the object based on the corrected biosignal BS.

In addition, the biosignal processing apparatus 200 may be implemented as an electronic apparatus such as a cellular phone (mobile phone), a smartphone, a laptop computer, a digital broadcasting terminal, a personal digital assistant (PDA), a portable multimedia player (PMP), a navigation system, an electronic tag, a lighting apparatus, a remote controller, or a wearable apparatus, or may be implemented as a computing apparatus, a distributed computing apparatus, a server apparatus, or the like which has at least one processor.

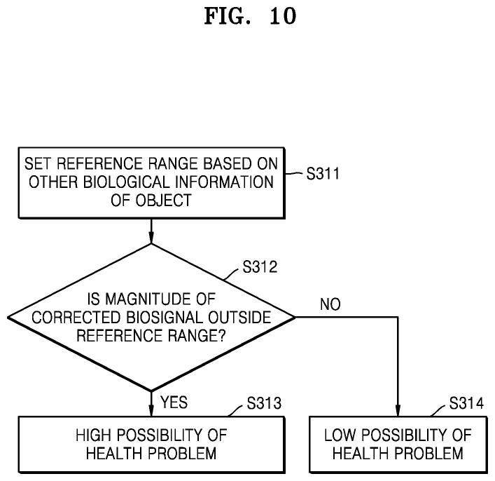

FIG. 10 is a flowchart illustrating a method of determining a health state according to an example embodiment. The method shown in FIG. 10 may be performed by the determining unit 230 shown in FIG. 8 to determine, for example, whether the heart has a problem.

Referring to FIG. 10, the determining unit 230 may set a reference range based on other biological information on an object (S311). For example, the reference range may include the magnitude of a biosignal determined to be of a healthy state. In other words, the reference range may include the magnitude of an electrocardiogram signal determined to be normal. The reference range may include a maximum value and a minimum value.

For example, the determining unit 230 may set the reference range based on fixed biological information and/or variable biological information. The reference range may be set by a statistical method. For example, as described with reference to FIG. 9, the trained health state determination algorithm may set the reference range based on fixed biological information and/or variable biological information on the object.

The determining unit 230 may determine whether the magnitude of a corrected biosignal is within or outside the reference range (S312). When the magnitude of the corrected biosignal is less than the minimum value of the reference range or greater than the maximum value of the reference range, the determining unit 230 may determine that the magnitude of the corrected biosignal is outside the reference range, that is, less than or greater than the reference range. When the magnitude of the corrected biosignal is greater than or equal to the minimum value of the reference range and less than or equal to the maximum value of the reference range, the determining unit 230 may determine that the magnitude of the corrected biosignal is within the reference range.

When the magnitude of the corrected biosignal is outside the reference range, the determining unit 230 may determine that the object probably has a health problem (S313), and when the magnitude of the corrected biosignal is within the reference range, the determining unit 230 may determine that the object unlikely has a health problem (S314).

For example, based on variable biological information such as a heart rate and the magnitude of a corrected electrocardiogram signal, the determining unit 230 may determine whether the heart has a problem. Based on an increasing rate of heartbeat and an increasing slope of the magnitude of the corrected electrocardiogram signal, the determining unit 230 may determine whether the heart has a problem. Based on an increasing rate of heartbeat, the determining unit 230 may set a reference range for an increasing slope of the magnitude of an electrocardiogram signal, and when an increasing slope of the magnitude of a corrected electrocardiogram signal is outside the reference range, the determining unit 230 may determine that the heart probably has a problem.

In another example, the determining unit 230 may set a reference range based on fixed biological information such as the height, weight, body fat, age, or gender of the object, and when the magnitude of the corrected electrocardiogram signal is outside the reference range, the determining unit 230 may determine that the heart probably has a problem.

In an embodiment, when it is determined that the possibility of health abnormality is high, the biosignal processing apparatus 200 may display a signal or information indicating the state, or may transmit, to a user's person terminal, a message recommending a health improving activity such as exercise or a medical checkup.

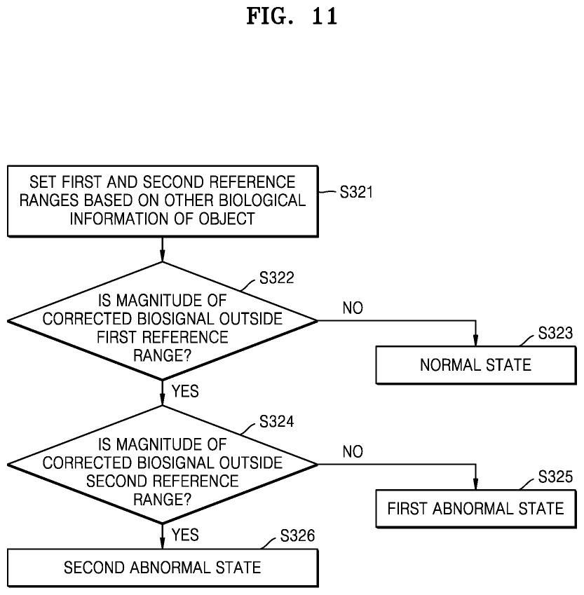

FIG. 11 is a flowchart illustrating a method of determining a health state according to an example embodiment. The method shown in FIG. 11 may be performed by the determining unit 230 shown in FIG. 8 to determine, for example, whether the heart has a problem.

Referring to FIG. 11, the determining unit 230 may set a first reference range and a second reference range based on other biological information on an object (S321). For example, the first reference range may include the magnitude of a biosignal determined to be normal (no health problem), and the second reference range may include the magnitude of a biosignal determined as indicating a future health problem. Each of the first reference range and the second reference range may include a maximum value and a minimum value. As described above, the determining unit 230 may set the first reference range and the second reference range by a statistical method based on fixed biological information and/or variable biological information.

The determining unit 230 may determine whether the magnitude of a corrected biosignal is within or outside the first reference range (S322). The determining unit 230 may compare the magnitude of the corrected biosignal, for example, the magnitude of a corrected electrocardiogram signal, with the maximum and minimum values of the first reference range to determine whether the magnitude of the corrected biosignal is within or outside the first reference range.

The determining unit 230 may determine the health state of the object as a normal state when the magnitude of the corrected biosignal is not outside the first reference range (S323). In other words, the determining unit 230 may determine that the possibility of a heart problem is low.

When the magnitude of the corrected biosignal is outside the first reference range, the determining unit 230 may determine whether the magnitude of the corrected biosignal is within or outside the second reference range (S324). When the magnitude of the corrected biosignal is not outside the second reference range, the determining unit 230 may determine the health state of the object as a first abnormal state (S325). The first abnormal state may refer to a state expected to have a health problem in the future, that is, a state expected to have a heart problem in the future, thereby requiring a health promotion for the object to prevent the future health problem.

When the magnitude of the corrected biosignal is outside the second reference range, the determining unit 230 may determine the health state of the object as a second abnormal state (S326). The second abnormal state may refer to a state in which the possibility of having a health problem is high, for example, a state requiring a medical checkup such as an electrocardiogram image checkup.

In an embodiment, when it is determined that the health state of the object is the first abnormal state, a biosignal processing apparatus (such as the biosignal processing apparatus 200 shown in FIG. 8) may display a notification signal or notification information or may transmit a message recommending a health promotion such as exercise to a user's personal terminal. When it is determined that the health state of the object is the second abnormal state, that is, when it is determined that the possibility of having a health problem is high, the biosignal processing apparatus 200 may display a warning signal or warning information or may transmit a message recommending a medical checkup such as an electrocardiogram image checkup to the user's personal terminal.

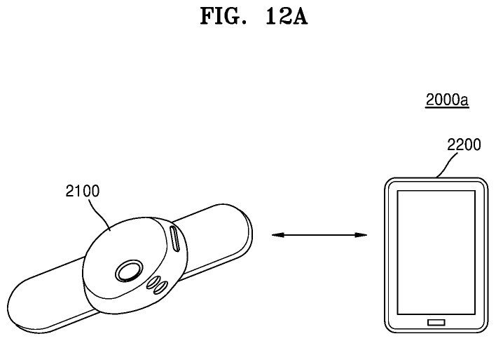

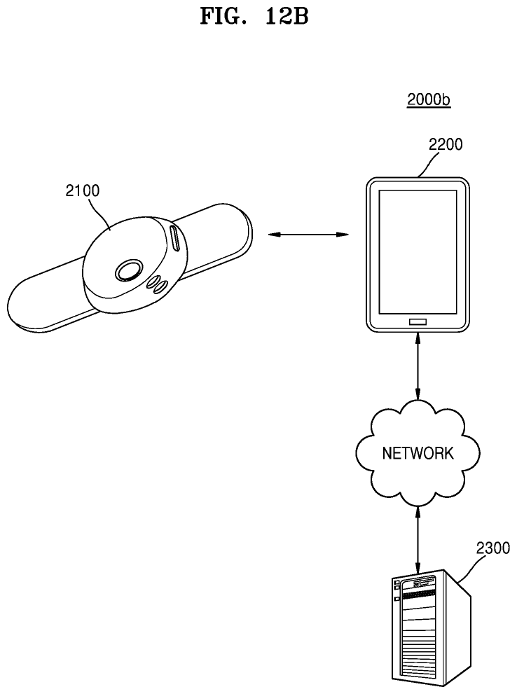

FIGS. 12A and 12B illustrate biosignal monitoring systems 2000a and 2000b according to embodiments.

Referring to FIGS. 12A and 12B, each of the biosignal monitoring systems 2000a and 2000b may include a biosignal measuring apparatus 2100 and a data receiving apparatus 2200, and the biosignal monitoring system 2000b may further include a management server 2300.

The biosignal measuring apparatus 2100 may be implemented as a module including the components described with reference to FIG. 2 and may be attached to an object to sense and generate a biosignal, for example, an electrocardiogram signal, by using a plurality of electrodes. As described above, the biosignal measuring apparatus 2100 may include an impedance measuring circuit configured to measure the impedance between the plurality of electrodes. Therefore, the biosignal measuring apparatus 2100 may generate a biosignal and an impedance value.

The biosignal measuring apparatus 2100 may communicate with the data receiving apparatus 2200 by a wired or wireless communication method. FIGS. 12A and 12B illustrate that the biosignal measuring apparatus 2100 directly communicates with the data receiving apparatus 2200. However, embodiments are not limited thereto. For example, the biosignal measuring apparatus 2100 may communicate with the data receiving apparatus 2200 through a repeater (not shown).

The biosignal measuring apparatus 2100 may provide a biosignal and an impedance value to the data receiving apparatus 2200. Alternatively, the biosignal measuring apparatus 2100 may correct the biosignal based on the impedance value and may provide the corrected biosignal to the data receiving apparatus 2200.

Examples of the data receiving apparatus 2200 may include electronic apparatus such as a cellular phone (mobile phone), a smartphone, a laptop computer, a digital broadcasting terminal, a personal digital assistant (PDA), a portable multimedia player (PMP), a navigation system, an electronic tag, a lighting apparatus, a remote controller, or a wearable apparatus. In addition, examples of the data receiving apparatus 2200 may include a computing apparatus, a distributed computing apparatus, a server apparatus, or the like which has at least one processor. Although the data receiving apparatus 2200 is illustrated as an electronic apparatus including a display, the data receiving apparatus 2200 may not include a display. The biosignal processing apparatus 200 described with reference to FIG. 8 may be implemented as the data receiving apparatus 2200.

The data receiving apparatus 2200 may receive a biosignal and an impedance value from the biosignal measuring apparatus 2100 and may correct the magnitude of the biosignal based on the impedance value to generate the magnitude of a corrected biosignal such as a corrected electrocardiogram signal.

Based on the magnitude of the corrected biosignal and other fixed and/or variable biological information, the data receiving apparatus 2200 may determine the health state of the object. The health state determining method described with reference to FIGS. 9 to 11 may be used in the current embodiments.

As shown in FIG. 12B, the data receiving apparatus 2200 may communicate with the management server 2300 through a network to transmit, to the management server 2300 through the network, data including a biosignal and an impedance value received from at least one biosignal measuring apparatus 2100; data including the magnitude of a corrected biosignal obtained by correcting the magnitude of a biosignal based on an impedance value; or data indicating the health state of the object. For example, the management server 2300 may manage data received from the data receiving apparatus 2200 in association with the object. For example, the management server 2300 may store and manage electrocardiogram data and other biological data such as fixed biological information and/or variable biological information in relation to the account of each object.

The apparatuses, units, circuits and/or modules described above may be implemented as hardware components, software components, and/or combinations of hardware components and software components. For example, apparatuses and components described in the embodiments may each be implemented using, for example, a processor, a controller, an arithmetic logic unit (ALU), a digital signal processing unit, a microcomputer, a field programmable gate array (FPGA), a programmable logic unit (PLU), or a microprocessor, or may each be implemented using any other device capable of executing instructions and responding to instructions, for example, using one or more general-purpose or special purpose computers. The processing apparatus may execute an operating system (OS) and one or more software applications executed on the operating system. The processing apparatus may also access, store, manipulate, process, and generate data in response to execution of software. In some parts of the present disclosure, the use of one processing apparatus is described for ease of understanding. However, those of ordinary skill in the art will understand that the processing apparatus may include a plurality of processing elements and/or a plurality of types of processing elements. For example, the processing apparatus may include a plurality of processors or one processor and one controller. In addition, other processing configurations such as parallel processors are also possible.

Software may include a computer program, code, instructions, or a combination of one or more thereof, which may constitute the processing apparatus to operate the processing apparatus as desired or may independently or collectively give commands to the processing apparatus. Software and/or data may be permanently or temporarily embodied in any kind of machine, component, physical device, virtual equipment, computer storage medium or device, or transmitted signal waves such that the software and/or data may be interpreted by the processing apparatus or may provide instructions or data to the processing apparatus. Software may be distributed over networked computer systems and may be stored or executed in a distributed manner. Software and data may be stored in one or more computer-readable recording media.

The methods described in the embodiments may be implemented in the form of program instructions that may be executed through various computers and may be recorded in a computer-readable medium. The computer-readable medium may include program instructions, data files, data structures, or a combination thereof. Program instructions specially designed and configured for embodiments or known and available to those skilled in the art of computer software may be recorded on the computer-readable medium. Examples of the computer-readable recording medium include: magnetic media such as hard disks, floppy disks, and magnetic tapes; optical media such as CD-ROMs and DVDs; magneto-optical media such as floppy disks; and hardware devices such as ROMs, RAMs, and flash memories specially configured to store and execute program instructions. Examples of program instructions include not only machine language codes such as those generated by a compiler, but also high-level language codes that may be executed by a computer using an interpreter or the like. The hardware devices described above may be configured to operate as one or more software modules to perform operations in embodiments, and vice versa.

Although some embodiments have been described with reference to the accompanying drawings, those of ordinary skill in the art may make various modifications and changes in the embodiments. For example, the techniques described above may be performed in an order different from the described order, and/or the components described above such as systems, structures, apparatuses, and circuits may be coupled to or combined with each other in manners different from the manners described above or may be replaced or substituted with other components or equivalents.

Therefore, other implementations, other embodiments, and equivalents may be provided without departing from the spirit and scope of the disclosure as defined by the following claims.

What is claimed is:

1. A biosignal measuring apparatus comprising:
a plurality of electrodes configured to be in contact with a skin of an object and receive electrical signals generated from the object;
a biosignal sensing circuit electrically connected to the plurality of electrodes and configured to generate a biosignal based on the electrical signals received through the plurality of electrodes;
an impedance measuring circuit electrically connected to the plurality of electrodes and configured to measure an impedance between the plurality of electrodes which is to be used for correcting a magnitude of the biosignal varying with time; and
a signal processing unit configured to receive the biosignal from the biosignal sensing circuit and an impedance value from the impedance measuring circuit, and correct the magnitude of the biosignal based on the impedance value.

2. The biosignal measuring apparatus of claim 1, wherein
the biosignal sensing circuit is configured to output the biosignal, which has a first waveform in a first period of an entire sensing period and a second waveform in a second period after the first period, and
the impedance measuring circuit is configured to output a first impedance value in the first period and a second impedance value in the second period.

3. The biosignal measuring apparatus of claim 2, wherein the signal processing unit is configured to correct a magnitude of the first waveform of the biosignal based on the first impedance value, and a magnitude of the second waveform of the biosignal based on the second impedance value.

4. The biosignal measuring apparatus of claim 1, wherein the impedance measuring circuit is configured to measure an impedance corresponding to a frequency band of the biosignal.

5. The biosignal measuring apparatus of claim 1, wherein the signal processing unit is configured to correct, based on an impedance value measured in a period in which the biosignal is measured, the magnitude of the biosignal which varies with time during an entire sensing period.

6. A biosignal processing apparatus comprising:
a receiving unit configured to receive, from a biosignal measuring apparatus, a biosignal generated based on electrical signals received through a plurality of electrodes attached to an object and an impedance value between the plurality of electrodes; and
a biosignal correcting unit configured to generate a corrected biosignal by correcting a magnitude of the biosignal based on the impedance value.

7. The biosignal processing apparatus of claim 6, wherein
the biosignal correcting unit is configured to correct a magnitude of a first waveform of the biosignal based on a first impedance value measured in a first period in which the first waveform of the biosignal is sensed, and
the biosignal correcting unit is configured to correct a magnitude of a second waveform of the biosignal based on a second impedance value measured in a second period in which the second waveform of the biosignal is sensed.

8. The biosignal processing apparatus of claim 6, further comprising a determining unit configured to determine a health state of the object based on a magnitude of a corrected biosignal and other biological information on the object.

9. The biosignal processing apparatus of claim 8, wherein the determining unit is configured to determine the health state of the object based on the magnitude of the corrected biosignal and at least one selected from the group consisting of height, weight, body fat, age, and gender of the object.

10. The biosignal processing apparatus of claim 8, wherein the determining unit is configured to determine the health state of the object based on the magnitude of the corrected biosignal and at least one selected from the group consisting of a heart rate, a breathing rate, a water content, a blood pressure, and an activity state measured from the object.

11. The biosignal processing apparatus of claim 8, wherein
when the magnitude of the corrected biosignal or a change in the magnitude of the corrected biosignal is outside a first reference range set by considering the other biological information and is within a second reference range set by considering the other biological information, the determining unit is configured to determine that the object is in a first abnormal state requiring a health promotion, and
when the magnitude of the corrected biosignal or the change in the magnitude of the corrected biosignal is outside the second reference range, the determining unit is configured to determine that the object is in a second abnormal state requiring a medical checkup.

12. The biosignal processing apparatus of claim 6, wherein the biosignal comprises an electrocardiogram signal.

13. A method of operating a biosignal processing apparatus, the method comprising:
receiving, from a biosignal measuring apparatus, a biosignal generated based on electrical signals received through a plurality of electrodes attached to a skin of an object and an impedance value between the plurality of electrodes; and
generating a corrected biosignal by correcting a magnitude of the biosignal based on the impedance value.

14. The method of claim 13, wherein the generating of the corrected biosignal comprises:
correcting a magnitude of a first waveform of the biosignal based on a first impedance value measured in a first period in which the first waveform of the biosignal is sensed; and
correcting a magnitude of a second waveform of the biosignal based on a second impedance value measured in a second period in which the second waveform of the biosignal is sensed.

15. The method of claim 13, further comprising determining a health state of the object based on other biological information on the object and a magnitude of the corrected biosignal.

16. The method of claim 15, wherein the determining of the health state of the object comprises determining the health state of the object based on the magnitude of the corrected biosignal and at least one selected from the group consisting of height, weight, body fat, age, and gender of the object.

17. The method of claim 15, wherein the determining of the health state of the object comprises determining the health state of the object based on the magnitude of the corrected biosignal and at least one selected from the group consisting of a heart rate, a breathing rate, a water content, a blood pressure, and an activity state measured from the object.

18. The method of claim 15, wherein the determining of the health state of the object comprises:
setting a reference range based on the other biological information; and
when the magnitude of the corrected biosignal or a change in the magnitude of the corrected biosignal is outside the reference range, determining that the object probably has a health problem.

19. The method of claim 15, wherein the determining of the health state of the object comprises:
setting a first reference range and a second reference range based on the other biological information;
when the magnitude of the corrected biosignal or a change in the magnitude of the corrected biosignal is outside the first reference range and is within the second reference range, determining that the object is in a first abnormal state requiring a health promotion; and
when the magnitude of the corrected biosignal or the change in the magnitude of the corrected biosignal is outside the second reference range, determining that the object is in a second abnormal state requiring a medical checkup.
